(19) United States
(12) Patent Application Publication
(10) Pub. No.: US 2006/0241473 A1
(43) Pub. Date: Oct. 26, 2006

(54) Ultrasonic probe and producing method therefor

(75) Inventor: Toshiaki Kuniyasu, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/370,022

(22) Filed: Mar. 8, 2006

(30) Foreign Application Priority Data
Mar. 9, 2005 (JP) 2005-066076

(51) Int. Cl. A61B 8/14

(52) U.S. Cl. 600/459

(57) ABSTRACT

Ultrasonic transducers disposed at a head of an ultrasonic probe are joined to a flexible sheet having a curved surface shape. Through holes are formed in the flexible sheet. The through hole is filled with conductive paste for electrically connecting to an individual electrode of the ultrasonic transducer. The flexible sheet is attached to a semicircular support. A surface of the support is provided with device-side terminals for electrically connecting to the conductive paste. The inside of the support is provided with wiring for connecting the device-side terminal to a wiring cable, which is connected to an ultrasonic observing unit.

### BACKGROUND OF THE INVENTION

1. Field of the Invention The present invention relates to an ultrasonic probe comprising ultrasonic transducers for applying ultrasonic waves to a relevant part of a biologic body and for receiving echo signals from the biologic body, and the present invention further relates to a producing method for the ultrasonic probe.

2. Description of the Related Art

In the medical field of recent years, medical diagnostics utilizing ultrasound images is put to practical use. The ultrasound image is obtained by electrically detecting echo signals, which are sent from a biologic body, with an ultrasonic observing unit connected to an ultrasonic probe via a connector. The ultrasonic observing unit applies ultrasonic waves to a relevant part of the biologic body from the ultrasonic probe. As to a mode for driving the ultrasonic probe, is known an electronic scan mode in which a plurality of ultrasonic transducers are disposed to transmit and receive the ultrasonic waves. In this mode, the ultrasonic transducers to be driven are selectively changed by electronic switches and so forth.

Regarding the ultrasonic probe of the electronic scan mode, there is a convex electronic scan mode in which the ultrasonic transducers (a number of which is 94 to 128, for example) are disposed at the top of the probe in a fan-like form. Beside this mode, there is a radial electronic scan mode in which the ultrasonic transducers (a number of which is 360, for example) are disposed at the periphery of the top of the probe. Further, these modes are classified into a one-dimensional array type and a two-dimensional array type in accordance with arrangement manners of the ultrasonic transducers.

With respect to methods for producing the ultrasonic transducers of the one-dimensional array type, various methods are proposed. In one of the proposed methods, a piezoelectric element is joined to a flexible backing material so as to interpose a flexible circuit board (see Japanese Patent Laid-Open Publication No. 7-327299). In another of the proposed methods, a piezoelectric element is attached to a flexible backing material and a flexible circuit board is joined to terminals of individual electrodes formed on an end portion of the piezoelectric element (see Japanese Patent Laid-Open Publication No. 8-89505).

As to the ultrasonic transducers produced by the technology described in the Publication No. 7-327299, there is a possibility that interference is caused between the piezoelectric element and wiring of the flexible circuit board to generate noises in a signal passing through the wiring. In the meantime, as to the ultrasonic transducers produced by the technology described in the Publication No. 8-89505, a space is necessary for forming the terminal on the end portion. Thus, there arises a problem in that it is impossible to prevent a size from enlarging. In addition, the technologies described in the above-noted Publications are unsuitable for producing the ultrasonic transducers of the two-dimensional array type.

### SUMMARY OF THE INVENTION

In view of the foregoing, it is a primary object of the present invention to provide an ultrasonic probe in which a compact and high-density ultrasonic transducer array is easily mounted.

It is a second object of the present invention to provide a producing method for an ultrasonic probe in which a compact and high-density ultrasonic transducer array is easily mounted.

In order to achieve the above and other objects, the ultrasonic probe according to the present invention comprises ultrasonic transducers disposed at a head of the ultrasonic probe in an array form. The ultrasonic transducers are joined to a flexible sheet having a curved surface shape. In the flexible sheet, through holes are formed. The though hole is filled with a conductive member for electrically connecting to an individual electrode of the ultrasonic transducer.

In a preferred embodiment, the flexible sheet is attached to a support having a curved surface shape. For instance, the support is formed in a semicircular shape including a convex shape and a concave shape, and a cylindrical shape. A surface of the support is provided with a terminal for electrically connecting to the conductive member, and the inside of the support is provided with wiring for connecting the terminal to a wiring cable electrically connecting to an ultrasonic observing unit.

In another embodiment, the flexible sheet is attached to a flexible wiring substrate, a surface of which is provided with a terminal for electrically connecting to the conductive member. Further, the inside of the flexible wiring substrate is provided with wiring for connecting the terminal to a wiring cable connecting to an ultrasonic observing unit. It is preferable that the flexible wiring substrate is attached to a support having a curved surface shape. For instance, the support is formed in a semicircular shape including a convex shape and a concave shape, and a cylindrical shape.

It is preferable that the conductive member is an adhesive which is applied to the flexible sheet when the ultrasonic transducers are attached to the flexible sheet. Alternatively, the conductive member may be a metal pin.

A method for producing the above-mentioned ultrasonic probe comprises the steps of forming the through holes in the flexible sheet and filling the through hole with the conductive member for electrically connecting to the individual electrode of the ultrasonic transducer. The ultrasonic-probe producing method further comprises the steps of joining a wafer of a piezoelectric element, which constitutes the ultrasonic transducer, to the flexible sheet, and dicing the wafer in an array form, and curving the flexible sheet.

According to the ultrasonic probe and the producing method therefor of the present invention, the ultrasonic transducers are joined to the flexible sheet having the through hole filled with the conductive member for electrically connecting to the individual electrode of the ultrasonic transducer. Further, the flexible sheet is curved. Thus, it is possible to easily mount the ultrasonic transducers of an array form having a compact curved shape and high-density curved surface.

### BRIEF DESCRIPTION OF THE DRAWINGS

The above objects and advantages of the present invention will become apparent from the following detailed description of the preferred embodiments of the invention when read in conjunction with the accompanying drawings, in which:

FIG. 1 is an enlarged sectional view showing a structure of a head of an ultrasonic probe according to the present invention;

FIG. 2 is an explanatory illustration showing arrangement of ultrasonic transducers of the ultrasonic probe;

FIG. 3 is an enlarged sectional view showing a structure of the ultrasonic transducer;

FIGS. 4A to 4D are illustrations showing a producing sequence of the ultrasonic probe, wherein FIG. 4A shows a process for forming through holes, FIG. 4B shows a process for applying conductive paste, FIG. 4C shows a process for joining a wafer of the ultrasonic transducer, and FIG. 4D shows a process for dicing the wafer of the ultrasonic transducer;

FIG. 5 is an enlarged sectional view showing a structure of a head of an ultrasonic probe using metal pins as conductive members;

FIGS. 6A to 6E are illustrations showing a producing sequence of the ultrasonic probe using the metal pins, wherein FIG. 6A shows a process for forming through holes, FIG. 6B shows a process for fitting the pin into the through hole, FIG. 6C shows a process for applying conductive paste, FIG. 6D shows a process for joining a wafer of the ultrasonic transducer, and FIG. 6E shows a process for dicing the wafer of the ultrasonic transducer;

FIG. 7 is a sectional view showing an embodiment in that a flexible wiring substrate is used; and

FIG. 8 is a sectional view showing an ultrasonic probe of a radial electronic scan mode.

### DETAILED DESCRIPTION OF THE PREFERRED EMBODIMENT(S)

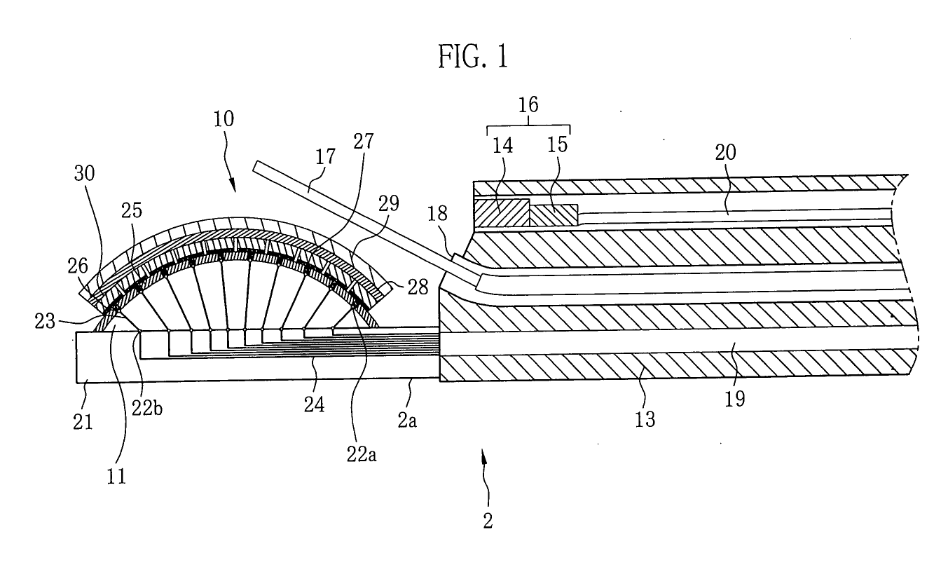

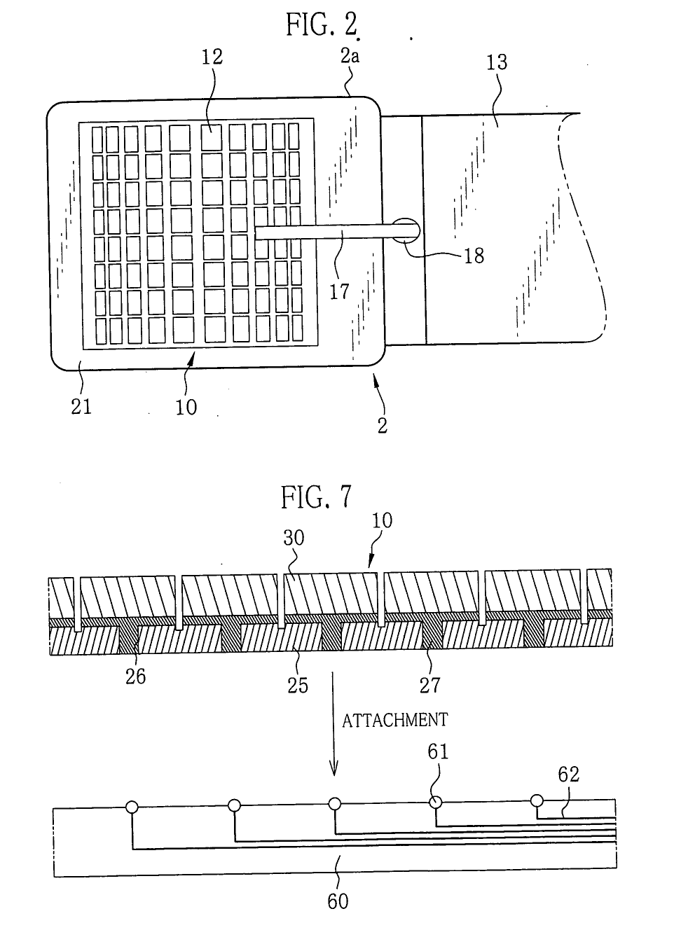

In FIGS. 1 and 2, an ultrasonic probe 2 according to the present invention is provided with an ultrasonic transducer array 10 disposed at a head 2a of the ultrasonic probe 2. The ultrasonic transducer array 10 adopts so-called convex electronic scan mode in which ultrasonic transducers 12 are arranged on a semicircular support 11 in a two-dimensional array form, such as explanatorily shown in FIG. 2.

An imaging device 16 is disposed at an upper portion of a sheath 13 connected to the head 2a. The imaging device 16 comprises an objective optical system 14 for receiving image light of a body part to be observed, and a CCD 15 for taking the image light to output image signals. A middle portion of the sheath 13 is provided with a channel 18 into which a puncture needle 17 is inserted. Further, wiring cables 19 and 20 are inserted into the sheath 13 so as to interpose the channel 18 used for the puncture needle 17. The wiring cable 19 electrically connects the ultrasonic transducer array 10 to an ultrasonic observing unit (not shown), and the wiring cable 20 electrically connects the imaging device 16 to the ultrasonic observing unit.

The support 11 is placed on a base 21 of the head 2a. The support 11 is made of a material having stiffness, which is a hard rubber and so forth and in which ultrasonic-wave attenuation material is added as need arises. The support 11 may have a concave shape.

The front and the rear of the support 11 are respectively provided with device-side terminals 22a and cable-side terminals 22b by a number of the ultrasonic transducers 12. Moreover, the inside of the support 11 is provided with wiring 23 for connecting the device-side terminal 22a and the cable-side terminal 22b. A conductive paste 27 described later is electrically connected to the device-side terminal 22a. Meanwhile, wiring 24 extending from the wiring cable 19 to the inside of the base 21 is electrically connected to the cable-side terminal 22b.

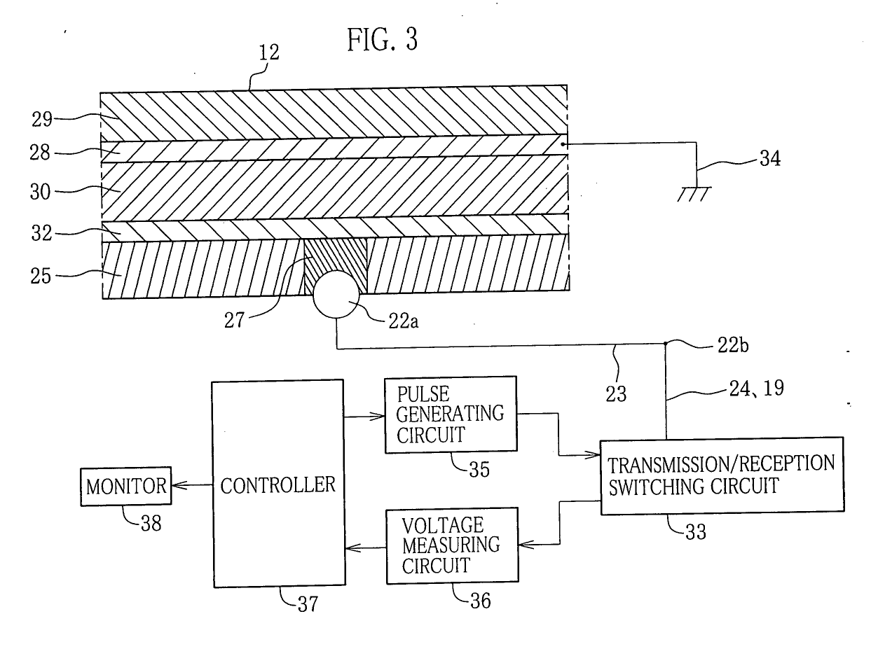

The ultrasonic transducer array 10 is joined to a flexible sheet 25 via which this array 10 is attached to the support 11. Through holes 26 are formed in the flexible sheet 25 and are filled with the conductive paste 27, which is applied to the flexible sheet 25 when the ultrasonic transducer array 10 is joined to the flexible sheet 25. By the way, for the purpose of avoiding complication, hatching is not drawn relative to the support 11 and the base 21. Although illustration is abbreviated, interspace formed between the ultrasonic transducers 12 is loaded with a filler of epoxy resin. Incidentally, reference numeral 28 denotes a metal film being as a common electrode (see FIG. 3) of the ultrasonic transducers 12, and reference numeral 29 denotes an acoustic matching layer, which is for matching acoustic impedance with a biologic body.

In FIG. 3, the ultrasonic transducer 12 comprises a piezoelectric element 30 and the acoustic matching later 29 of epoxy resin, which are located in this order from the flexible sheet 25. The piezoelectric element 30 comprises a thin film of PZT (lead zirconate titanate) and is interposed between an individual electrode 32 and the common electrode 28.

The individual electrode 32 is connected to a transmission/reception switching circuit 33 of the ultrasonic observing unit via the conductive paste 27 of the through hole 26, the device-side terminal 22a, the wiring 23, the cable-side terminal 22b, the wiring 24 and the wiring cable 19. Meanwhile, the common electrode 28 is grounded via wiring 34. In fact, such as described above, the common electrode 28 is the metal film superposed on the entire surfaces of the piezoelectric elements 30.

The transmission/reception switching circuit 33 changes transmission and reception of the ultrasonic waves, which are performed by the ultrasonic transducer 12, at predetermined time intervals. This circuit 33 is connected to a pulse generating circuit 35 and a voltage measuring circuit 36. The pulse generating circuit 35 applies pulse voltage to the piezoelectric element 30 when to emit the ultrasonic waves from the ultrasonic transducer 12 (when to transmit the ultrasonic waves). In virtue of this, the ultrasonic transducer 12 generates the ultrasonic wave having predetermined frequency.

The voltage measuring circuit 36 measures a voltage generated by the piezoelectric element 30 when the ultrasonic transducer 12 has received an echo signal from the biologic body (when the ultrasonic wave has been received). The voltage measuring circuit 36 sends a result of this measurement to a controller 37 in which the measurement result sent from the voltage measuring circuit 36 is converted into an ultrasonic image. The converted ultrasonic image is displayed on a monitor 38.

When obtaining in vivo ultrasonic images, an insert portion of the ultrasonic probe 2 is inserted into the biologic body. While optical images obtained by the imaging device 16 are observed with an endoscopic monitor, a relevant part is searched inside the biologic body. After the head 2a has reached the relevant part, an instruction is given to obtain the ultrasonic image. Upon this instruction, the ultrasonic wave is emitted from the ultrasonic transducer 12 in accordance with the pulse voltage, which is applied from the pulse generating circuit 35, to scan the biologic body while the transmission/reception switching circuit 33 switches the ultrasonic transducer 12 to transmit and receive the ultrasonic wave.

The echo signal from the biologic body is received by the ultrasonic transducer 12, and the voltage generated in the piezoelectric element 30 is measured by the voltage measuring circuit 36. The measurement result of the voltage measuring circuit 36 is sent to the controller 37 and is converted into the ultrasonic image therein. The converted ultrasonic image is displayed on the monitor 38. In addition, while the optical image or the ultrasonic image is observed, the puncture needle 17 is operated, if necessary, to take the relevant part of the biologic body.

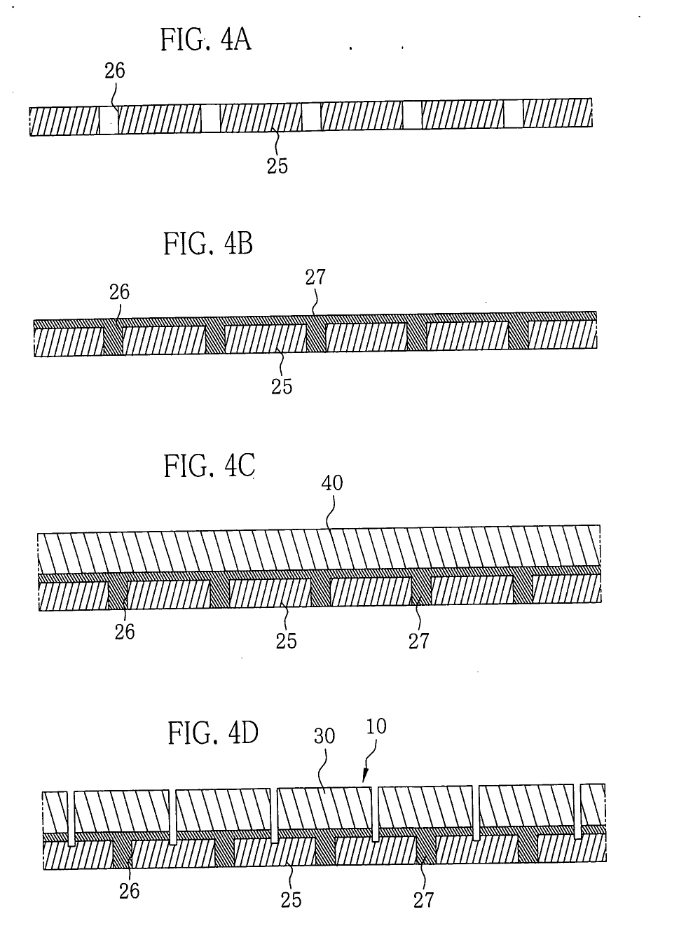

Next, a process for producing the ultrasonic probe 2 having the above structure is described below, referring to FIG. 4. First of all, as shown in FIG. 4A, the through holes 26 are formed in predetermined positions of the flexible sheet 25 by means of a laser, a punch, a drill and so forth. Then, as shown in FIG. 4B, the conductive paste 27 is screen-printed on the flexible sheet 25 by using a squeeze. In virtue of this, the through hole 26 is filled with the conductive paste 27.

After the conductive paste 27 has been screen-printed on the flexible sheet 25, a wafer 40 of the piezoelectric element 30 is joined to the flexible sheet 25 via the conductive paste 27, such as shown in FIG. 4C. Successively, as shown in FIG. 4D, the wafer 40 is diced in a two-dimensional array form. At this time, the conductive paste 27 is divided so as to correspond to each of the diced piezoelectric elements 30. Thus, the piezoelectric elements 30 are isolated from each other. Incidentally, the divided conductive past 27, which confronts the piezoelectric element 30, is regarded as the individual electrode 32.

After that, the flexible sheet 25 is bent so as to fit a curved surface shape of the support 11 and is attached to the support 11. Thereupon, the conductive paste 27 is electrically connected to the device-side terminal 22a disposed at the surface of the support 11. After filling the interspaces of the piezoelectric elements 30 with the filler, the metal film being as the common electrode 28 is attached to the surfaces of the piezoelectric elements 30. Finally, the acoustic matching layer 29 is attached to the common electrode 28 to complete the ultrasonic probe 2.

As described above in detail, the ultrasonic transducers 12 are joined to the flexible sheet 25 in which the through holes 26 are formed. The through hole 26 is filled with the conductive paste 27 electrically connecting to the individual electrode 32 of the ultrasonic transducer 12. Further, the surface shape of the flexible sheet 12 is curved. Thus, it is possible to easily mount the ultrasonic transducers 12 of the two-dimensional array form having the compact curved shape and the high-density curved surface.

The surface of the support 11 is provided with the device-side terminal 22a for electrically connecting to the conductive paste 27, and the inside of the support 11 is provided with the wiring 23 for connecting the device-side terminal 22a to the wiring cable 19. It is prevented that noises are added to the signals passing through the wiring 23. In virtue of this, receiver sensitivity of the ultrasonic wave becomes good so that the ultrasonic image of high quality is obtained.

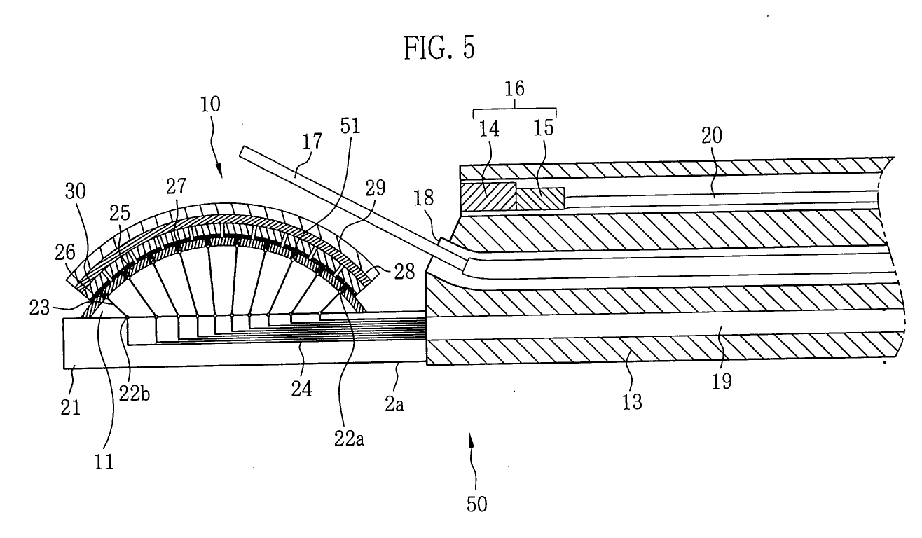

In the above embodiment, the conductive paste 27 is used as the conductive member. However, such as an ultrasonic probe 50 shown in FIG. 5, a metal pin 51 may be used instead of the conductive paste 27. In FIG. 5, hatching is not drawn relative to the support 11 and the base 21 similarly to FIG. 1.

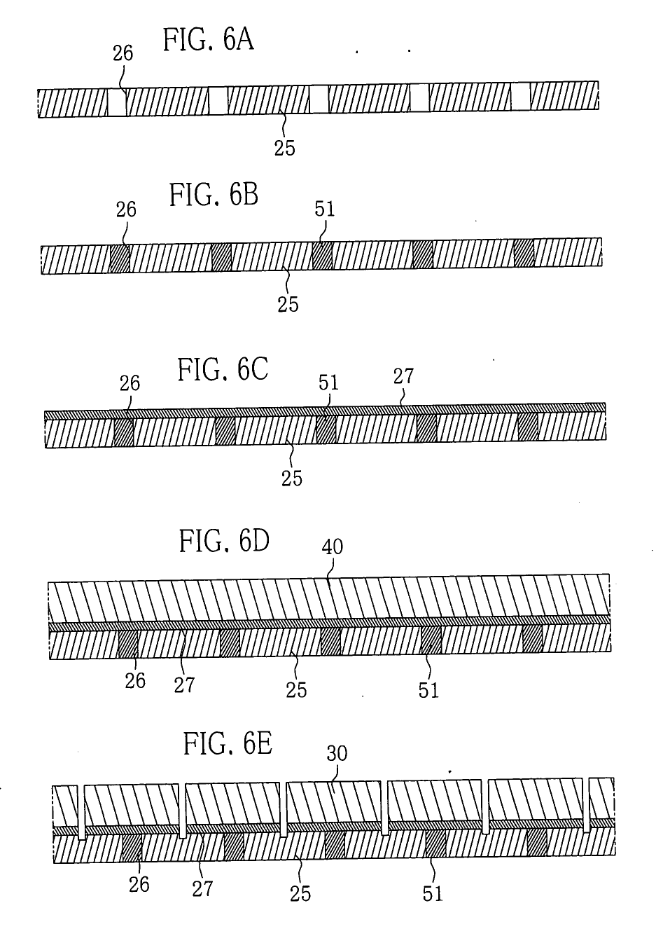

In this embodiment, a process for producing the ultrasonic probe 50 is as shown in FIGS. 6A to 6E. First of all, such as shown in FIG. 6A, the through holes 26 are formed in predetermined positions of the flexible sheet 25 similarly to the foregoing embodiment. After that, such as shown in FIG. 6B, the pin 51 is fitted into the through hole 26. Successively, such as shown in FIG. 6C, the conductive paste 27 is screen-printed on the flexible sheet 25 similarly to the foregoing embodiment. And then, such as shown in FIG. 6D, the wafer 40 of the piezoelectric element is joined to the flexible sheet 25. Finally, such as shown FIG. 6E, the wafer 40 is diced in a two-dimensional array form and the conductive paste 27 is divided so as to correspond to each of the diced piezoelectric elements 30. Thus, the piezoelectric elements 30 are isolated from each other. By the way, in a case using a commercial anisotropic conductive sheet into which metal pins are fitted in advance, the processes shown in FIGS. 6A and 6B are omitted. In this case, the flexible sheet 25 is attached to the support 11 after joining the wafer 40 of the piezoelectric element to the flexible sheet 25, and then the wafer 40 is diced in the two-dimensional array form. At this time, the anisotropic conductive sheet is completely divided every diced piezoelectric element 30 to isolate the piezoelectric elements 30 from each other.

Instead of providing the support 11 with the device-side terminal 22a and the wiring 23, a flexible wiring substrate 60 shown in FIG. 7 maybe used. A surface of the flexible wiring substrate 60 is provided with terminals 61 for electrically connecting to the conductive paste 27 (or the pin 51). The inside of the flexible wiring substrate 60 is provided with wiring 62 for connecting the terminals 61 to the wiring cable 19. In this case, when producing the ultrasonic probe, the flexible sheet 25 to which the ultrasonic transducer array 10 is attached is joined to the flexible wiring substrate 60, and then, this wiring substrate 60 is mounted on a support. Incidentally, a plurality of the flexible wiring substrates may be stacked to construct a multilayer form. In FIG. 7, hatching is not drawn relative to the flexible wiring substrate 60 for the similar reason with the support 11 and the base 21 shown in FIGS. 1 and 5.

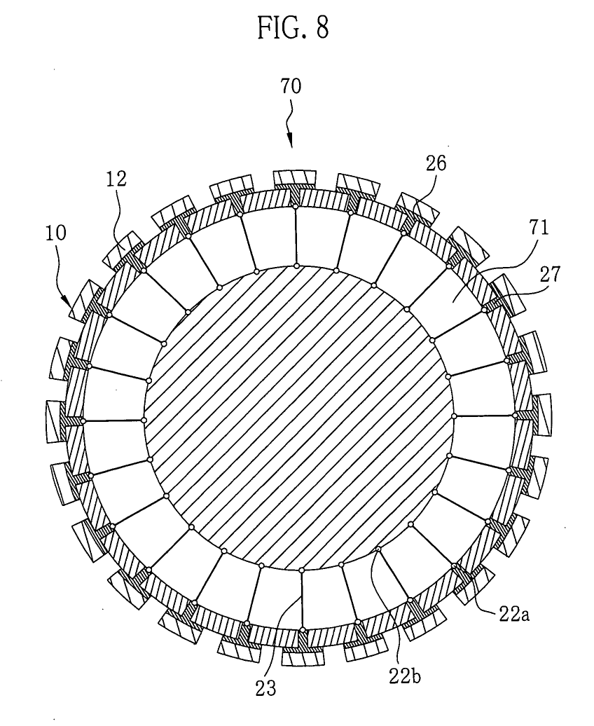

In the above embodiments, the ultrasonic transducer array 10 of the convex electronic scan mode is described. The present invention, however, may be adopted to an ultrasonic probe 70 of so-called radial electronic scan mode in which the ultrasonic transducers 12 are attached to a cylindrical support 71 via the flexible sheet 25 such as shown in FIG. 8.

Further, besides the ultrasonic transducer arrays 10 described in the above embodiments, the present invention may be adopted to an actuator for driving a focus lens and a zoom lens of a camera, a vibrating gyro to be used for an angular rate sensor, and the other transducer arrays.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. An ultrasonic probe, at a head of which a plurality of ultrasonic transducers are arranged in an array form, said ultrasonic probe comprising:
a flexible sheet to which said ultrasonic transducers are joined, said flexible sheet being bent so as to have a curved surface shape;
a through hole formed in said flexible sheet; and
a conductive member fitted into said through hole, said conductive member electrically connecting to an individual electrode of said ultrasonic transducer.

2. An ultrasonic probe according to claim 1, further comprising:
a support to which said flexible sheet is attached, said support having a curved surface shape.

3. An ultrasonic probe according to claim 2, wherein said support is formed in either of a semicircular shape and a cylindrical shape.

4. An ultrasonic probe according to claim 2, further comprising:
a terminal disposed at a surface of said support, said terminal electrically connecting to said conductive member; and
wiring led through the inside of said support, said wiring connecting said terminal to a wiring cable contained in said ultrasonic probe.

5. An ultrasonic probe according to claim 1, further comprising;
a flexible wiring substrate to which said flexible sheet is attached;
a terminal disposed at a surface of said flexible wiring substrate, said terminal electrically connecting to said conductive member; and
wiring led through the inside of said flexible wiring substrate, said wiring connecting said terminal to a wiring cable contained in said ultrasonic probe.

6. An ultrasonic probe according to claim 5, further comprising:
a support to which said flexible wiring substrate is attached, said support having a curved surface shape.

7. An ultrasonic probe according to claim 6, wherein said support is formed in either of a semicircular shape and a cylindrical shape.

8. An ultrasonic probe according to claim 1, wherein said conductive member is a conductive paste being as an adhesive applied to said flexible sheet at a time when said transducers are joined to said flexible sheet.

9. An ultrasonic probe according to claim 8, wherein said individual electrode of said ultrasonic transducer is said conductive paste applied to a surface of said flexible sheet.

10. An ultrasonic probe according to claim 1, wherein said conductive member is a metal pin.

11. A producing method for an ultrasonic probe, at a head of which a plurality of ultrasonic transducers are arranged in an array form, said producing method comprising the steps of:
forming through holes in a flexible sheet to be joined to said ultrasonic transducers;
fitting a conductive member into said through hole, said conductive member being for electrically connecting to an individual electrode of said ultrasonic transducer;
joining a wafer of a piezoelectric element of said ultrasonic transducer to said flexible sheet; and
dicing said wafer in said array form.

12. A producing method for the ultrasonic probe according to claim 11, further comprising the steps of:
bending said flexible sheet so as to have a curved surface shape; and
attaching said flexible sheet to a support having a curved surface shape.

13. A producing method for the ultrasonic probe according to claim 12, wherein said support is formed in either of a semicircular shape and a cylindrical shape.

14. A producing method for the ultrasonic probe according to claim 12, wherein a surface of said support is provided with a terminal for electrically connecting to said conductive member, and the inside of said support is provided with wiring for connecting said terminal to a wiring cable contained in said ultrasonic probe.

15. A producing method for the ultrasonic probe according to claim 11, further comprising the step of:
attaching said flexible sheet to a flexible wiring substrate, a surface of said flexible wiring substrate being provided with a terminal for electrically connecting to said conductive member, and the inside of said flexible wiring substrate being provided with wiring for connecting said terminal to a wiring cable contained in said ultrasonic probe.

16. A producing method for the ultrasonic probe according to claim 15, further comprising the step of:
bending said flexible wiring substrate so as to have a curved surface shape; and
attaching said flexible wiring substrate to a support having a curved surface shape.

17. A producing method for the ultrasonic probe according to claim 16, wherein said support is formed in either of a semicircular shape and a cylindrical shape.

18. A producing method for the ultrasonic probe according to claim 11, wherein said conductive member is a conductive paste being as an adhesive applied to said flexible sheet at a time when said transducers are joined to said flexible sheet.

19. A producing method for the ultrasonic probe according to claim 18, wherein said individual electrode of said ultrasonic transducer is said conductive paste applied to a surface of said flexible sheet.

20. A producing method for the ultrasonic probe according to claim 11, wherein said conductive member is a metal pin.
